(19) United States
(12) Patent Application Publication
(10) Pub. No.: US 2013/0158241 A1
(43) Pub. Date: Jun. 20, 2013

(54) Process for Preparing Crystalline 3,6,9-triaza-3,6,9-tris(carboxymethyl)-4-(4-ethoxybenzyl)undecanedioic Acid and Use for Production of Primovist®

(71) Applicants: Johannes Platzek, Berlin (DE); Wilhelm Trentmann, Munster (DE)

(72) Inventors: Johannes Platzek, Berlin (DE); Wilhelm Trentmann, Munster (DE)

(73) Assignee: BAYER INTELLECTUAL PROPERTY GMBH, Monheim (DE)

(21) Appl. No.: 13/703,579

(22) PCT Filed: Jun. 6, 2011
(86) PCT No.: PCT/EP11/59243

§ 371 Date: Feb. 25, 2013

(30) Foreign Application Priority Data
Jun. 11, 2010 (DE) 10 2010 023 890.2
Jul. 19, 2010 (BR) PII002466-2

(51) Int. Cl. C07C 227/18; C07C 229/26

(52) U.S. Cl. CPC C07C 227/18; C07C 229/26
USPC 534/16; 562/448

(57) ABSTRACT

The invention relates to a method for producing crystalline 3,6,9-triaza-3,6,9-tris(carboxymethyl)-4-(4-ethoxybenzyl)undecanedioic acid of the formula I

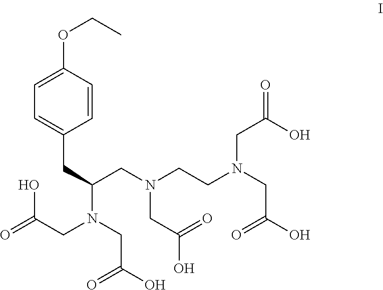 [SMILES: CCOc1ccc(C[C@@H](CN(CCN(CC(=O)O)CC(=O)O)CC(=O)O)N(CC(=O)O)CC(=O)O)cc1]

by saponifying 3,6,9-triaza-3,6,9-tris(tert-butoxycarbonylmethyl)-4-(4-ethoxybenzyl)-undecanedioic acid di-tert-butyl ester of the formula II in an aqueous alkali metal hydroxide solution and using 3,6,9-triaza-3,6,9-tris(carboxymethyl)-4-(4-ethoxybenzyl)undecanedioic acid of the formula I for producing the gadolinium complex of 3,6,9-triaza-3,6,9-tris(carboxymethyl)-4-(4-ethoxybenzyl)undecanedioic acid [(Gd-EOB-DTPA)=Primovist®].

This application is the U.S. National Phase of International Patent Application No. PCT/EP2011/059243, filed Jun. 6, 2011, which claims priority to European Patent Application No. 102010023890.2, filed Jun. 11, 2010, and Brazilian Patent Application No. PI1002466-2, filed Jul. 19, 2010, the disclosures of which are incorporated herein by reference in their entirety.

The invention relates to a method for producing crystalline 3,6,9-triaza-3,6,9-tris(carboxymethyl)-4-(4-ethoxybenzyl)undecanedioic acid of the formula I

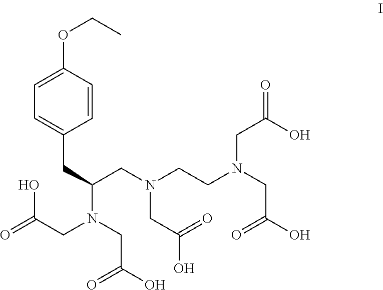 [SMILES: CCOc1ccc(C[C@@H](CN(CCN(CC(=O)O)CC(=O)O)CC(=O)O)N(CC(=O)O)CC(=O)O)cc1]

by saponifying 3,6,9-triaza-3,6,9-tris(tert-butoxycarbonylmethyl)-4-(4-ethoxybenzyl)-undecanedioic acid di-tert-butyl ester of the formula II,

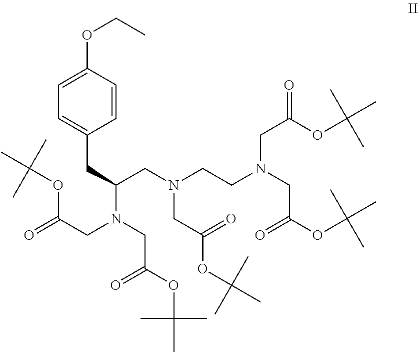 [SMILES: CCOc1ccc(C[C@@H](CN(CCN(CC(=O)OC(C)(C)C)CC(=O)OC(C)(C)C)CC(=O)OC(C)(C)C)N(CC(=O)OC(C)(C)C)CC(=O)OC(C)(C)C)cc1]

and to the use of 3,6,9-triaza-3,6,9-tris(carboxymethyl)-4-(4-ethoxybenzyl)undecanedioic acid of the formula I for producing the gadolinium complex of 3,6,9-triaza-3,6,9-tris(carboxymethyl)-4-(4-ethoxybenzyl)undecanedioic acid (Gd-EOB-DTPA=Primovist®).

3,6,9-Triaza-3,6,9-tris(carboxymethyl)-4-(4-ethoxybenzyl)undecanedioic acid (EOB-DTPA) is a complexing agent or chelator, the complexes of which with lanthanoids are used for producing agents for NMR and X-ray diagnosis, and also in radiation therapy. (EP 405 704 B1).

The gadolinium complex of 3,6,9-triaza-3,6,9-tris(carboxymethyl)-4-(4-ethoxybenzyl)-undecanedioic acid (Gd-EOB-DTPA) is known in the literature as a disodium salt under the names Eovist and Primovist® (gadoxetic acid)

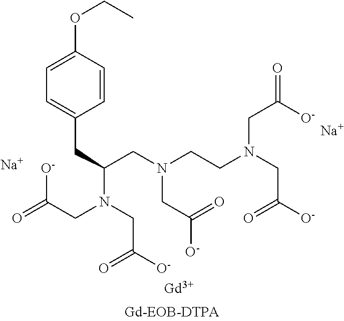

and has been permitted since 2004 as a liver-contrast agent as a contrast agent for nuclear spin tomography.

Primovist® is offered and used as a 0.25 molar solution as a contrast agent for parenteral use. The synthesis of the pure substance in a quality which can be used in injections (intravenous) formulations, in the known prior art, is very complex, expensive and requires chromatographic purification of the penta-tert-butyl ester of the formula II and subsequent saponification of the ester with trifluoroacetic acid and acidification of the reaction mixture with ion exchanger. The resultant monosodium salt is not crystalline and can only be obtained in solid form by freeze drying. The synthesis, incorporated herein by reference, is described in EP 0 405 704 B1 (Example 8) and in Schmitt-Willich H., Brehm M., Ewers C. L., Michl G., Muller-Fahrnow A., Petrov O., Platzek J., Raduchel B., Sulzle D. Synthesis and Physicochemical Characterization of a New Gadolinium Chelate: The Liver-Specific Magnetic Resonance Imaging Contrast Agent Gd-EOB-DTPA. Inorg Chem. 1999; 38(6): 1134-1144. This method, however, is unsuitable for production.

The actual production of Primovist® formulation (commercial product) consisted at the start in dissolving the previously freeze-dried gadolinium complex as a disodium salt in water, with addition of commercially conventional buffers, and also with addition of excess EOB-DTPA, generally in the form of the calcium complex of 3,6,9-triaza-3,6,9-tris(carboxymethyl)-4-(4-ethoxybenzyl)undecanedioic acid. The use of this excess of complexing agent (excess ligand) of or calcium(Ca) salt is discussed in detail in the patent EP 0 270 483 B2, incorporated herein by reference.

Since the gadolinium complex as a disodium salt has very hygroscopic properties, a so-called “upscaling” of this process is very difficult. Large-scale freeze dryers were used for this purpose which delivered the product in a relatively variable water content quality. Furthermore, the subsequent step of packaging and storing the drug substance is also very difficult. It would be more advantageous if a process were available in which the gadolinium complex could be produced from the ligand (EOB-DTPA) and gadolinium oxide directly. For this purpose, however, the availability of high-purity charges of the ligands (chelator=3,6,9-triaza-3,6,9-tris(carboxymethyl)-4-(4-ethoxybenzyl)undecanedioic acid) is a prerequisite.

It has now become possible to obtain the ligand in crystalline form in very high quality and yield without needing to use complex chromatographic and ion exchanger treatments. Intermediate isolation after freeze drying is dispensed with thereby.

It is an object of the invention to provide a process and thus EOB-DTPA qualities in which the gadolinium complex can be produced from the ligand (EOB-DTPA) and gadolinium oxide directly. For this purpose, however, the availability of high-purity ligands (=3,6,9-triaza-3,6,9-tris(carboxymethyl)-4-(4-ethoxybenzyl)undecanedioic acid) (EOB-DTPA) in sufficient quality and in a form which is storage-stable is essential.

The invention relates to a method for producing crystalline 3,6,9-triaza-3,6,9-tris(carboxymethyl)-4-(4-ethoxybenzyl)undecanedioic acid of the formula I, in which

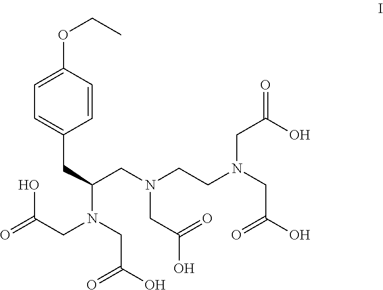 [SMILES: CCOc1ccc(C[C@@H](CN(CCN(CC(=O)O)CC(=O)O)CC(=O)O)N(CC(=O)O)CC(=O)O)cc1]

3,6,9-triaza-3,6,9-tris-(tert-butoxy-carbonylmethyl)-4-(4-ethoxybenzyl)undecanedioic acid-di-tert-butyl ester of the formula II

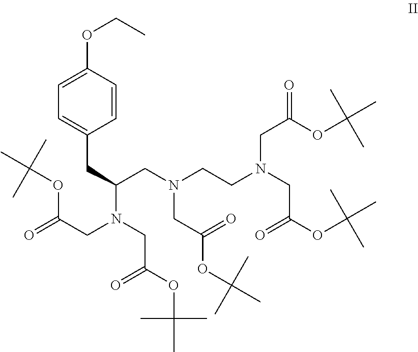 [SMILES: CCOc1ccc(C[C@@H](CN(CCN(CC(=O)OC(C)(C)C)CC(=O)OC(C)(C)C)CC(=O)OC(C)(C)C)N(CC(=O)OC(C)(C)C)CC(=O)OC(C)(C)C)cc1]

is hydrolysed with an aqueous alkali metal hydroxide solution, concentrated, the residue dissolved in water and the resultant solution acidified, or alternatively dissolved in a lower alcohol, hydrolysed with 5 to 7 equivalents of an aqueous alkali metal hydroxide solution at 50° C. to 90° C., the resultant reaction mixture is concentrated, the residue is dissolved in water and the resultant solution is acidified to a pH of 2.1 to 2.8, but preferably of 2.5 to 2.7 by slow addition of an aqueous inorganic acid, and filtered from the precipitate.

Under the method, it is not necessary to purify the penta-tert-butyl ester of the formula II; in addition, this method has the advantage that the process product occurs in crystalline form. The method can be carried out in such a manner that the ester of the formula II is dissolved in a lower alcohol, such as ethanol, n-propanol, isopropanol, or preferably methanol, is admixed with 5 to 7 equivalents of an 8 to 12 molar alkali metal hydroxide solution (preferably sodium hydroxide solution) and hydrolysed at the boiling temperature of the reaction mixture until completion of the reaction, which can be readily determined in a manner known per se by thin-layer chromatography (TLC) or gas chromatographic (GC) analysis.

After hydrolysis has been performed, the solvent is substantially removed, preferably by means of vacuum distillation, the residue dissolved in water and the resultant reaction mixture concentrated, the residue dissolved in water and the resultant solution is acidified to a pH of 2.1 to 2.8, but preferably of 2.5 to 2.7 by slow addition of an aqueous inorganic acid, preferably 12 to 25% strength sulphuric acid. The metered addition is performed in such a manner that the addition is interrupted at the start of turbidity and then continued with advancing crystallization. When the adjusted pH remains constant at 2.1 to 2.8, preferably at 2.5-2.7 after 12 hours, the crystals are filtered off. This crystal can be further purified by recrystallization from 4-8 times the amount of boiling water by means of crystallization, wherein it should be ensured that the cooling rate of the solution does not exceed a maximum of 10° C. per hour.

The ligand (EOB-DTPA) thus produced by means of the method according to the invention is not hygroscopic and is distinguished by very high purities (>98.75%, >99.0%) according to HPLC (100% method). The residual methanol solvent content of a product prepared by the process according to the invention, at <0.01%, is well below the specification limit (0.1%). It is likewise found that, as a result of the crystallizations, the enantiomeric excess (“e.e.”) is improved, thereby giving enantiomeric excesses of >99% e.e. The substance is very storage stable and can be processed further at a later time as required. The overall process has thus been greatly simplified, which is demonstrated by a reduction in cost since expensive chromatographic steps and also ion exchange desalting steps are no longer required. The technically difficult handling of freeze-dried material is also dispensed with.

The use of 3,6,9-triaza-3,6,9-tris(carboxymethyl)-4-(4-ethoxybenzyl)undecanedioic acid for producing the gadolinium complex of 3,6,9-triaza-3,6,9-tris(carboxymethyl)-4-(4-ethoxybenzyl)undecanedioic acid (Gd-EOB-DTPA) proceeds by reacting digadolinium trioxide in water and subsequent freeze drying, as described in DE 39 22 005 A, incorporated herein by reference, 1 and using crystalline 3,6,9-triaza-3,6,9-tris(carboxymethyl)-4-(4-ethoxybenzyl)undecanedioic acid of the formula I for producing a galenical formulation of the gadolinium complex of 3,6,9-triaza-3,6,9-tris(carboxym ethyl)-4-(4-ethoxybenzyl)undecanedioic acid (Gd-EOB-DTPA) for diagnostic purposes, particularly for MR tomography.

### EXAMPLES

### Example 1

### Crystalline 3,6,9-triaza-3,6,9-tris(carboxymethyl)-4-(4-ethoxybenzyl)undecanedioic acid

200 l of a methanolic solution of 3,6,9-triaza-3,6,9-tris(tert-butoxycarbonylmethyl)-4-(4-ethoxybenzyl)undecanedioic acid-di-tert-butyl ester (195 mol of crude ester from preliminary stage, without chromatographic purification, produced according to: Schmitt-Willich H., Brehm M., Ewers C. L., Michl G., Muller-Fahrnow A., Petrov O., Platzek J., Raduchel B., Sulzle D. Synthesis and Physicochemical Characterization of a New Gadolinium Chelate: The Liver-Specific Magnetic Resonance Imaging Contrast Agent Gd-EOB-DTPA. Inorg Chem. 1999; 38(6)) , incorporated herein by reference, are admixed with 280 l of methanol. The resultant solution is added to a solution of 45.1 kg (1130 mol) of sodium hydroxide and 121 l of water. The reaction mixture is heated for 2.5 hours under reflux and thereafter concentrated by evaporation to approximately 200 l under reduced pressure. The remaining oil is diluted with water to a weight of 397 kg. To the solution are added slowly dropwise 182 l of a 25% strength sulphuric acid (pH of the solution: 2.63). After the start of crystallization, the mixture is again adjusted to a pH of 2.6 by further addition of sulphuric acid. The reaction mixture is stirred for a further 12 hours at 20° C. The resultant crystals are filtered off and recrystallized from water. It is necessary in this case to maintain a cooling rate of a maximum of 10° C./h. After drying in a vacuum (50° C.), 74.7 kg of the 3,6,9-triaza-3,6,9-tris(carboxymethyl)-4-(4-ethoxybenzyl)undecanedioic acid are obtained in the form of colourless crystals. Yield: 68% of theory

Melting point 125° C. (decomposition). [a]_{D}²⁰=+8.2 (EtOH)

Analysis: C₂₃H₃₃N₃O₁₁. 4 H₂O

| | C | N | H |
|---|---|---|---|
| Reported | 46.07 | 7.01 | 6.89 |
| Found | 45.89 | 6.75 | 6.78 |

Purity (100% method, HPLC): >99%

Description of method (HPLC, 100% method)

Reagents
acetonitrile for chromatography
sulphuric acid, greater than 97%
tetrabutylammonium hydrogen sulphate
water
EOB-DTPA, working standard

### Test Method

The test method Related Substances/Degradation Products is combined with the test method Content. The test and control solutions must be prepared and aliquoted at the same temperature.

### Test Solutions P1 and P2

A solution with 1.00 mg/ml (0.95-1.05 mg/ml) of test substance is prepared by dissolving test substance in mobile phase A without heating, cP1/P2.

### Example

10.00 mg of test substance are dissolved without heating in mobile phase A in a 10 ml volumetric flask, and made up to the mark.

### Control Solution V

A solution with 1.00 mg/ml (corresponding to 0.95-1.05 mg/ml) of EOB-DTPA is prepared by dissolving at least 10 mg of EOB-DTPA, working standard, m, in mobile phase A in a volumetric flask with the volume V[V].

### Example

10.00 mg of EOB-DTPA, working standard are dissolved without heating in mobile phase A in a 10 ml volumetric flask, and made up to the mark.

### Test Conditions

Injection of test solution P: 10 μl
Injection of control solution V: 10 μl
Injection scheme: e.g. V, max. 3·P1 and P2, V
Detector: UV detector
Detector wavelength: 225 nm
Column: steel, length 12.5 cm, internal d=4.6 mm
Stationary phase: Hypersil ODS, 3 μm or equivalent
Mobile Phase A: 2 g of tetrabutylammonium hydrogen sulphate are dissolved in 900 ml of water for chromatography. Added to this solution are 100 ml of acetonitrile for chromatography. The pH is adjusted to 1.4 using 97% sulphuric acid. The mobile phase can be adapted with 5% of water or 2% of acetonitrile for chromatography. Different volumes of the mobile phase can be prepared, with a consistent concentration.
Mobile Phase B: acetonitrile for chromatography
Gradient programme:

| Time point | Flow rate | | |
|---|---|---|---|
| [min] | [ml/min] | A % (v/v) | B % (v/v) |
| 0 | 1.0 | 100 | 0 |
| 10 | 1.0 | 100 | 0 |
| 38 | 1.0 | 75 | 25 |
| 39 | 1.0 | 100 | 0 |
| 50 | 1.0 | 100 | 0 |

Temperature: room temperature
Data recording time: 50 minutes
System suitability test: The variation coefficient (VC) from at least 6 injections of the control solution V must be ≦1.0%.
   All peaks must be capable of integration.

### Example 2

Production of a 0.25 M Primovist formulation using crystalline 3,6,9-triaza-3,6,9-tris(carboxymethyl)-4-(4-ethoxybenzyl)undecanedioic acid (Tris-HCl buffer plus Ca complex excess)

56.0 g of calcium carbonate are dissolved in 1.344 kg of 3.6% strength aqueous hydrochloric acid and this solution is added to a suspension charged in advance consisting of 33.06 kg of crystalline 3,6,9-triaza-3,6,9-tris(carboxymethyl)-4-(4-ethoxybenzyl)undecanedioic acid, 14.944 kg of a 25% strength aqueous sodium hydroxide solution and 11.26 kg of gadolinium oxide and added to 160 l of water. The mixture is heated to 90° C. for approximately 2 h; in the course of this the gadolinium oxide dissolves until a clear solution is formed. Then, 301.66 g of Trometamol (Tris buffer) are added and the mixture is allowed to cool to 30° C. The pH is adjusted to pH 7.2 (selecting either a 5% aqueous HCl or a 5% aqueous sodium hydroxide solution). The total volume of the solution is adjusted to 250.8 l by adding water. The solution is filtered through a membrane (nitrogen pressure) and can then be charged into commercially conventional vials and sterilized.

Although the present invention has been described in detail in connection with the above embodiments and/or examples, it should be understood that such detail is illustrative and not restrictive, and that those skilled in the art can make variations without departing from the spirit of the invention. The invention is defined by the appended claims and all changes to the invention that fall within the meaning and the range of equivalency of the claims are to be embraced within their scope.

1) Method for producing crystalline 3,6,9-triaza-3,6,9-tris(carboxymethyl)-4-(4-ethoxybenzyl)undecanedioic acid of formula I, 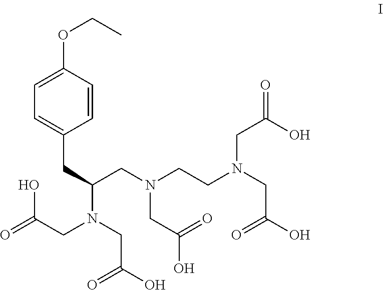 [SMILES: CCOc1ccc(C[C@@H](CN(CCN(CC(=O)O)CC(=O)O)CC(=O)O)N(CC(=O)O)CC(=O)O)cc1]
characterized in that 3,6,9-triaza-3,6,9-tris-(tert-butoxy-carbonylmethyl)-4-(4-ethoxy-benzyl)undecanedioic acid di-tert-butyl ester of formula II 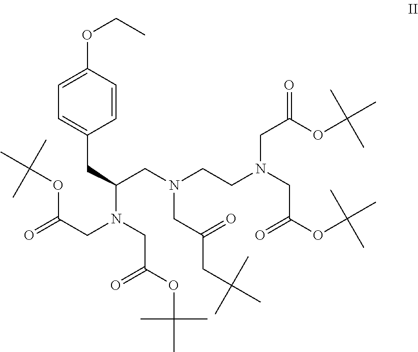 [SMILES: CCOc1ccc(C[C@@H](CN(CCN(CC(=O)OC(C)(C)C)CC(=O)OC(C)(C)C)CC(=O)CC(C)(C)C)N(CC(=O)OC(C)(C)C)CC(=O)OC(C)(C)C)cc1]
is hydrolysed with an aqueous alkali metal hydroxide solution, concentrated, a residue is dissolved in water and a resultant solution is acidified and filtered from a precipitate.

2) Method for preparing crystalline 3,6,9-triaza-3,6,9-tris(carboxymethyl)-4-(4-ethoxybenzyl)undecanedioic acid of formula I 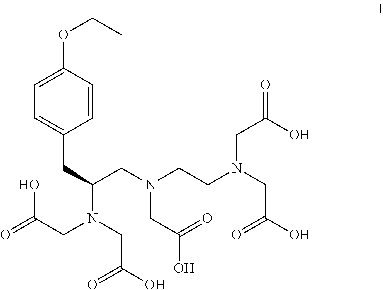 [SMILES: CCOc1ccc(C[C@@H](CN(CCN(CC(=O)O)CC(=O)O)CC(=O)O)N(CC(=O)O)CC(=O)O)cc1]
characterized in that 3,6,9-triaza-3,6,9-tris(tert-butoxycarbonylmethyl)-4-(4-ethoxybenzyl)undecanedioic acid di-tert-butyl ester of formula II 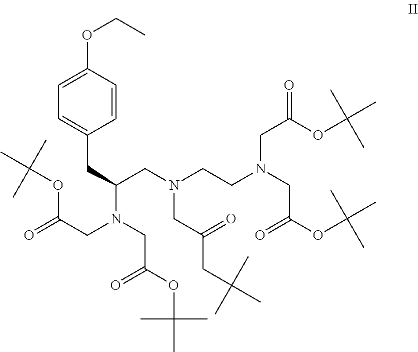 [SMILES: CCOc1ccc(C[C@@H](CN(CCN(CC(=O)OC(C)(C)C)CC(=O)OC(C)(C)C)CC(=O)CC(C)(C)C)N(CC(=O)OC(C)(C)C)CC(=O)OC(C)(C)C)cc1]
is dissolved in a lower alcohol, hydrolysed with an aqueous alkali metal hydroxide solution, a resultant reaction mixture is concentrated, a residue dissolved in water and a resultant solution acidified by slow addition of an aqueous inorganic acid and filtered from a precipitate.

3) Method for producing crystalline 3,6,9-triaza-3,6,9-tris(carboxymethyl)-4-(4-ethoxybenzyl)undecanedioic acid according to claim 1, characterized in that resultant crude crystals are dissolved in 4- to 6-times an amount of boiling water and then cooled with a cooling rate of a maximum of 10° C. per hour.

4) Use of 3,6,9-triaza-3,6,9-tris(carboxymethyl)-4-(4-ethoxybenzyl)undecanedioic acid of formula I 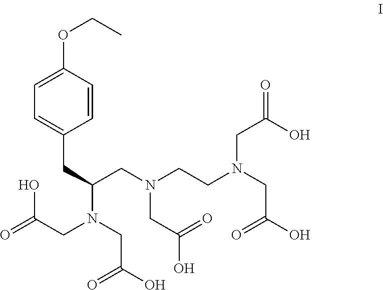 [SMILES: CCOc1ccc(C[C@@H](CN(CCN(CC(=O)O)CC(=O)O)CC(=O)O)N(CC(=O)O)CC(=O)O)cc1]
for producing a gadolinium complex of 3,6,9-triaza-3,6,9-tris(carboxymethyl)-4-(4-ethoxybenzyl)undecanedioic acid (Gd-EOB-DTPA).

5) Use of crystalline 3,6,9-triaza-3,6,9-tris(carboxymethyl)-4-(4-ethoxybenzyl)undecanedioic acid of formula I 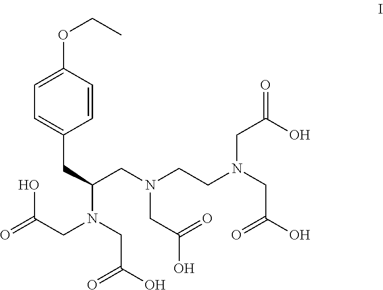 [SMILES: CCOc1ccc(C[C@@H](CN(CCN(CC(=O)O)CC(=O)O)CC(=O)O)N(CC(=O)O)CC(=O)O)cc1]
for producing a galenical formulation of the gadolinium complex of 3,6,9-triaza-3,6,9-tris(carboxymethyl)-4-(4-ethoxybenzyl)undecanedioic acid (Gd-EOB-DTPA).

6) 3,6,9-Triaza-3,6,9-tris(carboxymethyl)-4-(4-ethoxybenzyl)undecanedioic acid in a purity of greater than 99%.

7. Method for producing crystalline 3,6,9-triaza-3,6,9-tris(carboxymethyl)-4-(4-ethoxybenzyl)undecanedioic acid according to claim 2, characterized in that resultant crude crystals are dissolved in 4- to 6-times an amount of boiling water and then cooled with a cooling rate of a maximum of 10° C. per hour.
